(19) United States
(12) Patent Application Publication
(10) Pub. No.: US 2004/0158054 A1
(43) Pub. Date: Aug. 12, 2004

(54) Di-ribonucleotides as specific viral RNA-polymerase inhibitors for the treatment or prevention of viral infections

(76) Inventors: Jim Zhen Wu, Aliso Viejo, CA (US); Haoyun An, Carlsbad, CA (US); Zhi Hong, Aliso Viejo, CA (US)

Correspondence Address: ROBERT D. FISH; RUTAN & TUCKER, LLP, P.O. BOX 1950, 611 ANTON BLVD., 14TH FLOOR, COSTA MESA, CA, 92628-1950, US

(21) Appl. No.: 10/360,218

(22) Filed: Feb. 7, 2003

(51) Int. Cl. C07H 21/04

(52) U.S. Cl. 536/26.1

(57) ABSTRACT

Contemplated compounds and methods are directed towards a dinucleotide comprising a first and second nucleoside, wherein the dinucleotide inhibits a viral RNA polymerase, and wherein at least one of the nucleosides exhibits antiviral effect when cleaved from the dinucleotide.

### FIELD OF THE INVENTION

The field of the invention is enzyme inhibitors, and particularly polymerase inhibitors of viral polymerases, and their methods of use.

### BACKGROUND OF THE INVENTION

The concept of selected dinucleosidyl phosphodiester compounds as prodrugs for antineoplastic drugs was discovered in 1963, when bis(thioinosine)-5′,5′-phosphate was found to inhibit 6-mercaptopurine-resistant cancer cell lines (Montgomery et al., Nature 1963). This homodimeric prodrug form of 6-mercaptopurine (6-MP) riboside and 6-MP was shown to be 25- and 240-fold more potent against neoplastic cells than 6-MP riboside and 6-MP, respectively. Subsequently, various heterodimers of 5-fluorouracil deoxynucleotide (FUdR) and 6-MP were synthesized in which the nucleosides were coupled in a 3′-5′ or 5′-5′ fashion, and the resultant compounds were at least 200-fold more cytotoxic to mercaptopurine resistant cell lines than 6-MP alone. While various explanations for the increased potency were discussed, it is generally believed that the higher potency resulted from the release of FUdR and 6-MP ribonucleotide into the cells. Exemplary modes of coupling FUdR and 6-MP are shown in Prior Art FIG. 1.

Based on the early success, efforts were made to prepare various 3′-5′ and 5′-5′ dinucleotide prodrugs of Ara-C, FUdR, Ara-A, 6-MP in order to enhance cytotoxicity and deliver the drugs into the cells. Administration of these compounds to cell cultures had variable success and ranged from improved potency of such compounds to being entirely inactive. In 1988, this prodrug concept was introduced for antiviral drug design (Puech et al, J. Med. Chem, 1988; Shimizu et al, Nucleosides & Nucleotides, 1992). Various dinucleoside phosphodiesters, aryl phosphotriesters and methyl phosphonates of Ara-A, AZT, d4T, d4C were synthesized and evaluated against herpes simplex viruses and HIV. Compared to the simple combination of the corresponding monomeric nucleosides, some of the dinucleotide prodrugs showed synergistic potency with lower cytotoxicity, and the mechanism of action was attributed to slow intracellular hydrolysis of the dinucleotide to the respective nucleoside and nucleoside monophosphate by phosphodiesterase.

In still further examples, dinucleosidyl phosphotriesters were prepared for ddA and d4T. However, biological activity was significantly lower compared to the corresponding nucleosidyl phosphodiesters (Shimizu et al, Nucleosides Nucleotides 1992), which was attributed to the lack of phosphotriesterase activities in eukaryotic cells (a dinucleotide phosphotriester is believed to be dependent on a non-enzymatic process for conversion to the phosphodiester, which is then hydrolyzed to respective nucleosides).

Thus, although there are numerous dinucleoside and dinucleotide compounds known in the art, all or almost all of them suffer from one or more disadvantages. Therefore, there is still a need to provide improved compositions and methods for dinucleoside and dinucleotide compounds.

### SUMMARY OF THE INVENTION

The present invention is directed to compositions and methods of inhibition of viral polymerases, and especially viral RNA-dependent RNA polymerases, wherein particularly preferred compounds comprise a dinucleotide.

In one especially preferred aspect, contemplated dinucleotides include a first nucleoside that is covalently coupled to a second nucleoside, wherein the dinucleotide inhibits an RNA-dependent RNA polymerase of a virus (e.g., HCV) while the first nucleoside is covalently coupled to the second, and wherein the first nucleoside and/or the second nucleoside have an antiviral effect against the virus when the dinucleotide is cleaved into a first portion comprising the first nucleoside, and a second portion comprising the second nucleoside.

In further contemplated aspects, the first and/or second nucleoside may be covalently coupled to a phosphate (or modified phosphate) group, which is most typically bound to the 5′-OH group of the sugar to form the corresponding nucleotide(s). Additionally, or alternatively, the sugar in contemplated first and/or second nucleosides may be modified, and particularly preferred modifications include modifications at the 2′-atom (e.g., OCH₃, CH₃, CH₂OH) in alpha and/or beta orientation.

In yet another especially preferred aspect, contemplated dinucleotides will have a general structure according to Formula 1

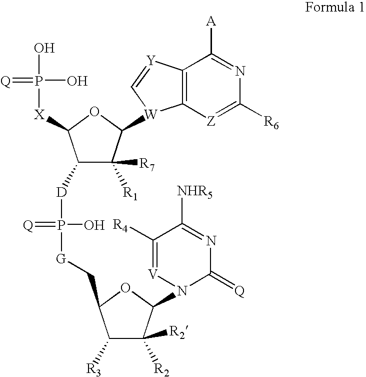

wherein A is H, OR, SR, NH₂, or NHR; V, W, Y, and Z are independently CH or N; Q is O or S; X is O, S, NR, CH₂ or null; D and G are independently null, CH₂, CF₂, O, S, or NH; R₁, R₂, R₂′, R₃ are independently H, OR, Halogen, CF₃, CCl₃, CHCl₂, CH₂OH, NO₂, CN, N₃, ═O, SR, NH₂, NHR, NHCOR, NHSO₂R, NHCONHR, or NHCSNHR; R₄ is R; R₅ is H, NH₂, NHR, NR₂, NHCOR, NHSO₂R, COR, SO₂R; R₆ is H, NH₂, NHCOR, NHSO₂R, NHNH₂, NHNHR, NHR, or NR₂; R₇ is H, OR, SR, Halogen, R, CF₃, CN, CHCl₂, CH₂OH, N₃, NH₂, or CH₂Cl; and wherein R is H or an optionally substituted alkyl, alkenyl, alkynyl, acyl, or aryl.

In still further preferred aspects, R₁, R₂, and R₃ are OH, wherein Q is O, and wherein D and G are O; and optionally at least one of R₂′ and R₇ is CH₃, and further optionally W, Y, and Z are N, V is CH, A is NH₂, and wherein R₄, R₅ and R₆ are H. Moreover, in contemplated dinucleotides, the 5′-phosphate group is replaced by an OH group, or may be modified such that the modification is preferentially removed from the dinucleotide in a hepatocyte (e.g., via a diester of the phosphate group).

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a prior art figure depicting known dinucleotides with antineoplastic activity.

FIG. 2 is a schematic representation of a contemplated mode of action of exemplary compounds according to the inventive subject matter.

FIG. 3 is a schematic representation of an exemplary synthetic route for some selected compounds according to the inventive subject matter.

FIG. 4 is a schematic representation of an exemplary synthetic route for other selected compounds according to the inventive subject matter.

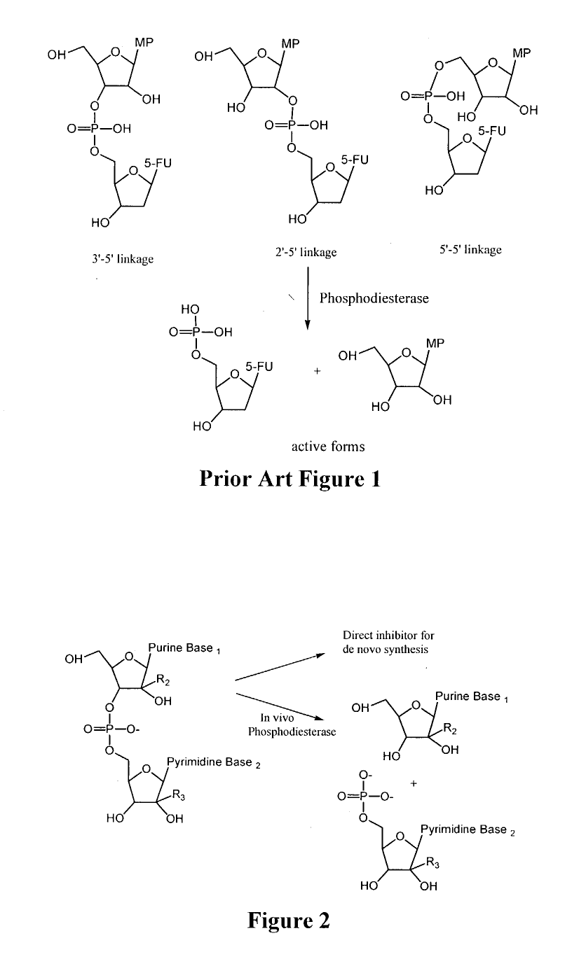

### DETAILED DESCRIPTION

The inventors discovered that contemplated dinucleotide and dinucleoside compounds and their analogs and prodrugs may act in a dual mode action, in which (a) the dinucleotide acts as a direct (RNA-dependent) RNA polymerase inhibitor by base pairing with the polymerase substrate, and (b) in which the dinucleotide acts as a prodrug for one, and more preferably for two antivirally active nucleotides (e.g., may provide a slow release of nucleotides before and after cellular uptake thereby reducing nucleoside metabolism and thus improving the therapeutic index).

Based on observations previously disclosed in co-pending provisional patent application with the serial No. 60/373,735 (filed Apr. 17, 2002, and which is incorporated by reference herein), the inventors discovered that various dinucleotides and their derivatives may have a dual mode of action, wherein such compounds may function as a direct inhibitor for an RNA polymerase, and especially for HCV NS5B, but may also serve as a prodrug for delivery of particularly desirable nucleotides to a cell infected with a virus, thereby exhibiting further antiviral effect.

The term “antiviral effect” as used herein refers to both direct and indirect effects, wherein direct antiviral effects include inhibition of a viral polymerase, inhibition of a viral nuclease, inhibition of viral protein processing, inhibition of viral priming activity, inhibition of viral protein assembly, and inhibition of viral entry and/or exit from a cell. Indirect antiviral effects include stimulation of the immune system to increase an immune response, and especially contemplated indirect antiviral effects include modulation of the Th1/Th2 balance (e.g., relative increase of Th1 over Th2, or vice versa), or stimulation of IFN-gamma or IL-12 secretion.

Recent work on the selectivity of initiating nucleotides by HCV NS5B (Shim, et al, J. Virology, 2002) indicates that a simple nucleoside or a dinucleotide (e.g., ApC without 5′-phosphate) is able to initiate de novo synthesis to at least some degree. As a primer for RNA replication, a dinucleotide is complementary to the 3′-end of the HCV (+) or (−) strands of RNA genome. The sequence of the last ten nucleotides of HCV (+) strand 3′-end is 5′-CAGAUCAAGU-3′, whereas for the (−) strand, it is 5′-UCGGGGGCUGGC-3′. Therefore, it is contemplated that dinucleotides based on AC or GC scaffold with suitable modification should be capable of binding to the NS5B productive complex and block the initiation step of de novo synthesis.

In particularly preferred aspects of the inventive subject matter, the 5′-phosphate of the dinucleotide may be omitted, which should greatly improve the compound's permeability. Alternatively, the 5′-phosphate of the dinucleotide may be modified to increase selectivity of delivery to a cell (e.g., hepatocyte) via prodrug formation (infra). It is further generally contemplated that dinucleotides according to the inventive subject matter may undergo various degradation and/or modification processes in vivo (e.g., hydrolysis, dephosphorylation, deamination, etc.). To stabilize the dinucleotide or the nucleotide/nucleoside and to reduce unfavorable metabolism in vivo, it is contemplated that modifications (e.g., addition of 2′-methyl (Walton et al, J. Am. Chem. Soc., 1966) or replacement of the 2′-hydroxyl group with the 2′-O-methyl group may be introduced into the dinucleotide.

Consequently, preferred dinucleotide inhibitors for an RNA polymerase, and especially for the HCV NS5B polymerase, will have a structure as illustrated in Structure 1A below. More preferably, contemplated compounds include those in which the first and second base comprise a purine or pyrimidine derivative, or any modified nucleoside base that is able to form a proper base pair with the corresponding base in the template strand of the viral RNA. Even more preferably, contemplated compounds will have a structure according to Structure 1B below in which most preferably X₂ and X₄=O, R₁ and R₄=OH or OMe, and R₂ and R₃=CH₃ to form 2′-methylAp2′-methylC.

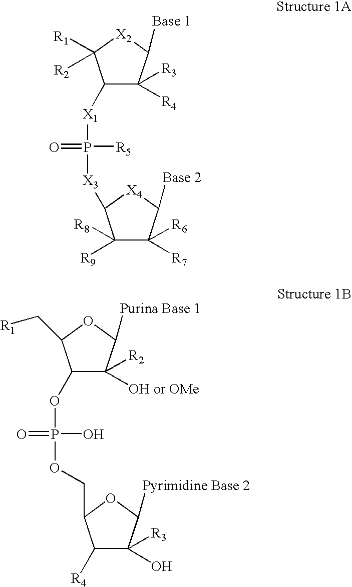

With respect to the “Base 1” or “Base 2” in compounds according to Structure 1A, it is generally contemplated that all compounds (a) in which a plurality of atoms (wherein at least one atom is an atom other than a carbon atom) form a ring via a plurality of covalent bonds and (b) that are able to base pair with the viral template strand through one, two, or three hydrogen bonds, or simply through hydrophobic interaction are considered suitable.

Particularly contemplated heterocyclic bases have between one and three rings, wherein especially preferred rings include 5- and 6-membered rings with nitrogen, sulfur, and/or oxygen as the non-carbon atom (e.g., imidazole, pyrrole, triazole, dihydropyrimidine). For example, suitable heterocyclic bases include the 1,2,4-triazole-3-carboxamide base from ribavirin or 1,2,4-triazole-3-carboxamidine base from viramidine. Further contemplated heterocycles may be fused (i.e., covalently bound) to another ring or heterocycle, and are thus termed “fused heterocycle” as used herein. Especially contemplated fused heterocycles include a 5-membered ring fused to a 6-membered ring (e.g., purine, pyrrolo[2,3-d]pyrimidine), and a 6-membered ring fused to another 6-membered or higher ring (e.g., pyrido[4,5-d]pyrimidine, benzodiazepine). Moreover, it should be recognized that suitable heterocyclic bases may further include one or more substituents, double and/or triple bonds, and any chemically reasonable combination thereof. It should also be appreciated that all of the contemplated heterocyclic bases may be coupled to contemplated sugars via a carbon atom or a non-carbon atom in the heterocyclic base.

Similarly, with respect to the “Purine Base 1” and “Pyrimidine Base 2” in compounds according to Structure 1B, it should be recognized that the same considerations as discussed above apply to the extent that (a) the “Purine Base 1” includes a purine or purine-type (i.e., nitrogen containing 5-membered ring fused to a nitrogen containing 6-membered ring) scaffold, and (b) the “Pyrimidine Base 1” includes a pyrimidine or pyrimidine-type (i.e., nitrogen containing 6-membered ring) scaffold.

R₁-R₄ and R₆-R₉ in compounds according to Structure 1A and R₁-R₄ in compounds according to Structure 1B may vary considerably, and it is generally contemplated that suitable substituents for those radicals independently include H, OH, CH₂OH, halogen, mono-, di-, and triphosphates/phosphonated/phosphoramidates/thiophosphates (preferably as C5′ esters), alkyl, alkenyl, alkynyl, aryl, alkaryl, alkoxy groups, acyl, amino groups and amines, nitrogen-containing substituents, sulfur-containing substituents, etc. Depending on the particular coupling between the two nucleosides/nucleotides, it should also be recognized that X₁, X₃, and R₅ may vary considerably. For example, where the coupling is via a regular phosphate diester, X₁, X₃, and R₅ are O. On the other hand where coupling is via a thiophosphate or phosphoamidate, X₁ and X₃ are O while R₅ is S or NH₂, respectively. Yet further contemplated alternative phosphate-type couplings include boranophosphates.

In still further contemplated aspects, the coupling may also include a spacer between at least one of the nucleoside/nucleotide and the phosphate group. Consequently, X₁ and X₃ may also independently include an alkyl, alkenyl, glycol, etc.

With respect to suitable sugars in contemplated dinucleotides, it should be recognized that suitable sugars may vary considerably, and that alternative sugars (i.e., sugars other than optionally modified ribofuranose) may include sugars in which the heteroatom in the cyclic portion of the sugar is an atom other than oxygen. Thus, X₂ and X₄ in compounds according to Structure 1A may vary considerably and suitable X₂ and X₄ atoms/groups include O, S, NH, CH₂, CHF or CF₂. Other alternative sugars may not be cyclic, but may be in a linear (open-chain) form. Moreover, suitable sugars may also include one or more double bonds, and all of the contemplated sugars may be in the D- or L-configuration. Numerous contemplated sugars and sugar analogs are commercially available. However, where contemplated sugars are not commercially available, it should be recognized that there are various methods known in the art to synthesize such sugars. For example, suitable protocols can be found in “Modern Methods in Carbohydrate Synthesis” by Shaheer H. Khan (Gordon & Breach Science Pub; ISBN: 3718659212), in U.S. Pat. Nos. 4,880,782 and 3,817,982, in WO88/00050, or in EP199,451.

Consequently, particularly preferred compounds according to the inventive subject matter will have a structure of Formula 1

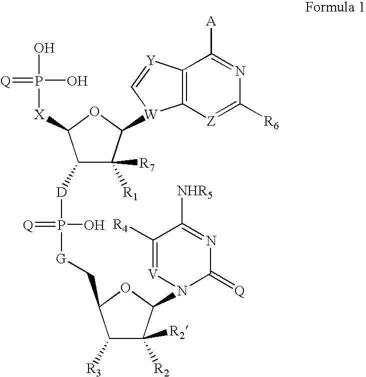

wherein A is H, OR, SR, NH₂, or NHR; V, W, Y, and Z are independently CH or N; Q is O or S; X is O, S, NR, CH₂ or null; D and G are independently null, CH₂, CF₂, O, S, or NH; R₁, R₂, R₂′, R₃ are independently H, OR, Halogen, CF₃, CCl₃, CHCl₂, CH₂OH, NO₂, CN, N₃, ═O, SR, NH₂, NHR, NHCOR, NHSO₂R, NHCONHR, or NHCSNHR; R₄ is R; R₅ is H, NH₂, NHR, NR₂, NHCOR, NHSO₂R, COR, SO₂R; R₆ is H, NH₂, NHCOR, NHSO₂R, NHNH₂, NHNHR, NHR, or NR₂; R₇ is H, OR, SR, Halogen, R, CF₃, CN, CHCl₂, CH₂OH, N₃, NH₂, or CH₂Cl; and wherein R is H or an optionally substituted alkyl, alkenyl, alkynyl, acyl, or aryl.

Where contemplated dinucleotides are employed as inhibitors for viral RNA-dependent RNA polymerases, and especially for the HCV NS5B polypeptide, it is particularly preferred that R₁, R₂, and R₃ are OH, wherein Q is O, and wherein D and G are O. In further especially preferred compounds, at least one of R₂′ and R₇ is CH₃, and W, Y, and Z are N, V is CH, A is NH₂, and R₄, R₅ and R₆ are H.

Exemplary contemplated dual mechanism of action is illustrated in FIG. 2, in which contemplated compounds may act as a direct inhibitor for a polymerase via hybridization with a viral template strand and in which contemplated compounds may act as a prodrug to deliver at least one (and more typically two) nucleosides/nucleotides with antiviral activity to a cell (e.g., via hydrolysis of the internucleotide linkage).

Depending on the particular desired functionality (i.e., either acting as direct inhibitor, as prodrug, or having both activities), it is contemplated that the compounds according to the inventive subject matter may be modified accordingly. For example, where the compound 2′-methylAp2′-methylC exhibits no or relatively low in vivo inhibitory effect against HCV NS5B, it is contemplated that the compound can be modified to increase in vivo stability of the internucleotide phosphodiester bond. Among other modifications, preferred strategies include replacement of the phosphodiester bond by a methyl ester (R₅=—OCH₃), other alkyl ester, aryl ester, H phosphonate (R₅=—H), phosphothioate (R₅=—S), phosphoamidate (R₅=—NH₂) or even an amino acid link.

On the other hand, it is also contemplated that the di-ribonucleotide 2′-methylAp2′-methylC may be cleaved by a phosphodiesterase in vivo into one or two active antiviral compounds (e.g., the respective nucleoside and nucleoside monophosphate), which may exert all or almost all of the antiviral effect. For example, both 2′-beta-methyl adenosine and 2′-methyl-beta-cytidine (which may be generated from 2′-methylAp2′-methylC) have been demonstrated to exhibit significant activity against HCV replication in vivo with an EC50 from 0.2 to 2 μM. However, it should also be recognized that a combination of the modes of action is considered particularly suitable, i.e., the dinucleotide compound exhibits an antiviral effect (e.g., via hybridization with viral template strand) as well as antiviral effect once the dinucleotide is cleaved into the first and second portion comprising the first and second nucleoside, respectively.

Where contemplated compounds are employed (at least in part) to deliver a nucleoside and/or nucleotide to a cell via a dinucleotide form, it is particularly preferred that the nucleoside or nucleotide in the 5′-position of the dinucleotide comprises a nucleoside that can be relatively efficiently phosphorylated, and that the nucleotide in the 3′-position of the dinucleotide comprises a nucleoside that is relatively inefficiently phosphorylated (since after hydrolysis, the 3′-nucleoside will be in the form of nucleoside monophosphate). The term “relatively efficiently phosphorylated” as used herein refers to in vitro phosphorylation of a nucleoside by a respective kinase with an efficiency of at least 30%, more typically at least 50%, and most typically at least 70% (measured as percent phosphorylated nucleoside of total nucleoside used after steady state is reached). Similarly, the term “relatively inefficiently phosphorylated” as used herein refers to in vitro phosphorylation of a nucleoside by a respective kinase with an efficiency of less than 30%, more typically less than 15%, and most typically less than 5%. Of course, it should be appreciated that the two nucleosides/nucleotides resulting from cleavage of the dinucleotide may exhibit additive or even synergistic effects (e.g., have distinct mechanism of action).

It should further be recognized that contemplated dinucleotides are particularly advantageous over presently known ribodinucleosides, and as such dinucleotides may suffer from a lack of stability due to the presence of 2′-hydroxyl group (RNA oligonucleotides are easily hydrolyzed due to the nucleophilic attack of the vicinal 2′-hydroxyl group to form a stable phosphotriester intermediate). Thus, addition of a 2′-substituent, and especially a 2′-methyl group or masking the 2′-hydroxyl group through alkylation or other modification may advantageously increase in vivo stability.

In yet another alternative aspect of preferred dinucleotides, it is further contemplated that stability of the dinucleotide may be increased by lengthening the linker between the two nucleosides such that the phosphorus atom will be less likely to be subject to enzymatic attack or nucleophilic attack by the 2′-hydroxyl group as illustrated in Structure 2 below.

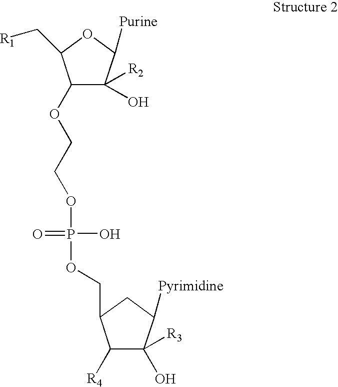

Contemplated Prodrugs and Metabolites

It should still further be appreciated that the compounds according to the inventive subject matter also include prodrug forms, phosphorylated forms (most preferably at the C5′-atom of the purine- or purine-type nucleoside in the dinucleotide) and/or metabolites of contemplated dinucleotides. Particularly suitable prodrug forms will include a moiety that is covalently coupled to at least one of the C2′-atom, C3′-atom, and C5′-atom of the purine nucleoside in the dinucleotide, thereby replacing the OH group, wherein the moiety is preferentially cleaved from the compound in a target cell (e.g., Hepatocyte) or a target organ (e.g., liver). While not limiting to the inventive subject matter, it is preferred that cleavage of the prodrug into the active form of the drug is mediated (at least in part) by a cellular enzyme, particularly receptor, transporter, and cytochrome-associated enzyme systems (e.g., CYP-system).

Where the purine-type nucleoside (or other nucleoside at the 5′-terminus of the dinucleotide) further comprises a phosphate group or analog of the phosphate group (e.g., thiophosphate or boranophosphate), it is especially contemplated that suitable prodrugs comprise a cyclic ester, ether, or other compound that forms a cyclic structure that includes the phosphorus atom (e.g., cyclic phosphate, cyclic phosphonate and/or a cyclic phosphoamidate), which is preferentially cleaved in a hepatocyte to produce the corresponding dinucleotide. There are numerous such prodrug forms known in the art, and all of those are considered suitable for use herein. However, especially contemplated prodrug forms are disclosed in WO 01/47935 (Novel Bisamidate Phosphonate Prodrugs), WO 01/18013 (Prodrugs For Liver Specific Drug Delivery), WO 00/52015 (Novel Phosphorus-Containing Prodrugs), and WO 99/45016 (Novel Prodrugs For Phosphorus-Containing Compounds), all of which are incorporated by reference herein. Consequently, especially suitable prodrug forms include those targeting a hepatocyte or the liver.

Still further particularly preferred prodrug forms include those described by Renze et al. in Nucleosides Nucleotides Nucleic Acids 2001 April-July;20(4-7):931-4, by Balzarini et al. in Mol Pharmacol 2000 November;58(5):928-35, or in U.S. Pat. No. 6,312,662 to Erion et al., U.S. Pat. No. 6,271,212 to Chu et al., U.S. Pat. No. 6,207,648 to Chen et al., U.S. Pat. No. 6,166,089 and U.S. Pat. No. 6,077,837 to Kozak, U.S. Pat. No. 5,728,684 to Chen, and published U.S. Application with the number 20020052345 to Erion, all of which are incorporated by reference herein. Alternative contemplated prodrug forms include those comprising a phosphate and/or phosphonate non-cyclic ester (SATE ester, pivaloyl ester, etc.), and an exemplary collection of suitable prodrug forms is described in U.S. Pat. No. 6,339,154 to Shepard et al., U.S. Pat. No. 6,352,991 to Zemlicka et al., and U.S. Pat. No. 6,348,587 to Schinazi et al. Still further particularly contemplated prodrug forms are described in FASEB J. 2000 September;14(12):1784-92, Pharm. Res. 1999, August 16:8 1179-1185, and Antimicrob Agents Chemother 2000, March 44:3 477-483, all of which are incorporated by reference herein.

Thus, particularly preferred prodrug forms will comprise a moiety covalently coupled to at least one of the C2′-atom, C3′-atom, and C5′-atom, wherein at least part of the moiety is preferentially cleaved from the compound in a target cell or target organ. As used herein, the term “preferentially cleaved . . . in a target cell or target organ” means that cleavage occurs in a particular target cell or target organ at a rate that is at least 3 times, more typically at least 10 times, and most typically at least 50 times higher than in a non-target cell or non-target organ. The term “target cell” or “target organ” as used herein refers to a cell or organ that is infected with a virus, and especially includes a hepatocyte infected with an HCV virus. Cleavage may be mediated by enzymes (but also by non-enzymatic processes, e.g., via reductive cleavage), and it is particularly preferred that enzymatic cleavage is mediated by a liver-specific enzyme system (e.g., CYP system). Consequently, it should be appreciated that certain prodrug forms of contemplated compounds may be cleaved in a target cell and/or target organ to provide a nucleotide analog. Alternatively, prodrugs may also be converted to the corresponding nucleoside (e.g., where the moiety does not include a phosphorus atom).

An exemplary preferred prodrug of contemplated compounds may therefore include a moiety according to Formula M1 or M2 (covalently coupled to the compound, typically to the C5′-atom, C2′-atom, and/or C3′-atom):

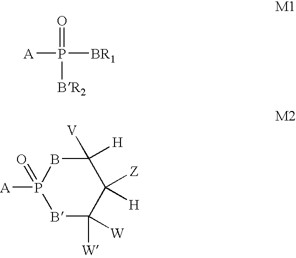

wherein A is O or CH₂ and replaces the 5′-OH group of the 5′-nucleoside of contemplated compounds. B and B′ are independently O or NH, and where in M1 B is NH then R₁ or R₂ is an amino acid that forms a peptide bond with the N atom of the NH. R₁, R₂, V, W, and W′ are independently hydrogen, alkyl, alkenyl, alkynyl, aryl, alkaryl, each of which is optionally substituted, and Z is hydrogen, CHWOH, CHWOCOW′, SW, or CH₂-aryl. Especially preferred compounds according to Formula M2 are those in which in A is O or CH₂, B and B′ are independently O or NH, in which Z, W, and W′ are H and V is m-chlorophenyl.

With respect to metabolites of contemplated compounds, it should be recognized that all metabolites are deemed suitable that have a desirable therapeutic effect, and especially an antiviral effect. Consequently, particularly suitable metabolites will generally include 5′-phosphates (e.g., monophosphate, diphosphate, and/or triphosphate esters), which may or may not be generated by an enzyme (e.g., kinase, oxidase). Further metabolites include those that are generated via enzymatic action on the heterocyclic base (e.g., via deaminase, deamidase, or hydroxylase). Thus, it should be recognized that metabolites of compounds according to the inventive subject matter may be generated in vivo by enzymatic and/or non-enzymatic reaction(s), wherein such reaction(s) may increase (e.g., via glycosylation, phosphorylation, amino transferase reaction, etc.) or decrease the molecular weight of contemplated compounds (e.g., by hydrolytic action, deamination, dephosphorylation, etc.).

Use of Contemplated Compounds

It is generally contemplated that compounds according to the inventive subject matter in their dinucleotide form (which may or may not include a 5′-phosphate group) will inhibit a viral polymerase. While not wishing to be bound to a particular theory or hypothesis, the inventors contemplate that such inhibition will at least in part be mediated by complementary binding of the dinucleotide to the viral template strand in, at, or near the active site of the viral polymerase. Additionally (or alternatively), the inventors contemplate that the compounds according to the inventive subject matter will comprise at least one nucleoside having biological activity, and especially contemplated biological activities include in vitro and in vivo inhibition of DNA and/or RNA polymerases, reverse transcriptases, and ligases. Thus, contemplated compounds will exhibit particular usefulness as in vitro and/or in vivo antiviral agents, antineoplastic agents, and/or immunomodulatory agents.

Especially contemplated antiviral activities include at least partial reduction of viral titers of respiratory syncytial virus (RSV), hepatitis B virus (HBV), hepatitis C virus (HCV), herpes simplex type 1 and 2, herpes genitalis, herpes keratitis, herpes encephalitis, herpes zoster, human immunodeficiency virus (HIV), influenza A virus, Hanta virus (hemorrhagic fever), human papilloma virus (HPV), yellow fever virus, and measles virus. The anti-HCV activity of the nucleosides and libraries were tested by replicon and BVDV cell-line based assays. The HCV NS5B polymerase activity was tested as described below, and were further tested for their capability of inhibition of replication of the hepatitis C virus in a cell-line based HCV replicon assay as described in V. Lohmann, F. Korner, J.-O. Koch, U. Herian, L. Theilmann, R. Bartenschlager, “Replication of a Subgenomic Hepatitis C virus RNAs in a Hepatoma Cell Line”, Sciences, 1999, 285, 110.

Especially contemplated immunomodulatory activity includes at least partial reduction of clinical symptoms and signs in arthritis, psoriasis, inflammatory bowel disease, juvenile diabetes, lupus, multiple sclerosis, gout and gouty arthritis, rheumatoid arthritis, rejection of transplantation, giant cell arteritis, allergy and asthma, but also modulation of some portion of a mammal's immune system, and especially modulation of cytokine profiles of Type 1 and Type 2. Where modulation of Type 1 and Type 2 cytokines occurs, it is contemplated that the modulation may include suppression of both Type 1 and Type 2, suppression of Type I and stimulation of Type 2, or suppression of Type 2 and stimulation of Type 1.

Where contemplated compounds are administered in a pharmacological composition, it is contemplated that suitable dinucleosides and/or dinucleotides can be formulated in admixture with a pharmaceutically acceptable carrier. For example, contemplated compounds can be administered orally as pharmacologically acceptable salts, or intravenously in a physiological saline solution (e.g., buffered to a pH of about 7.2 to 7.5). Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound in order to manage the pharmacokinetics of the present compounds for maximum beneficial effect in a patient.

In certain pharmaceutical dosage forms, prodrug forms of contemplated compounds may be formed for various purposes, including reduction of toxicity, increasing the organ or target cell specificity, etc. Among various prodrug forms, acylated (acetylated or other) derivatives, pyridine esters and various salt forms of the present compounds are preferred. One of ordinary skill in the art will recognize how to readily modify the present compounds to prodrug forms to facilitate delivery of active compounds to a target site within the host organism or patient. One of ordinary skill in the art will also take advantage of favorable pharmacokinetic parameters of the prodrug forms, where applicable, in delivering the present compounds to a targeted site within the host organism or patient to maximize the intended effect of the compound.

In addition, contemplated compounds may be administered alone or in combination with other agents for the treatment of various diseases or conditions. Combination therapies according to the present invention comprise the administration of at least one compound of the present invention or a functional derivative thereof and at least one other pharmaceutically active ingredient. The active ingredient(s) and pharmaceutically active agents may be administered separately or together and when administered separately this may occur simultaneously or separately in any order. The amounts of the active ingredient(s) and pharmaceutically active agent(s) and the relative timings of administration will be selected in order to achieve the desired combined therapeutic effect.

Experimental Data

### Synthesis of Exemplary Contemplated Compounds

It is generally contemplated that the compounds of the inventive subject matter may be prepared using numerous synthetic routes, and that all known routes are considered suitable for use herein. For example, while some synthetic routes will yield desired compounds one at a time, it should also be recognized that contemplated compounds may be prepared in parallel in a combinatorial library approach.

### Synthesis of 2′-methoxy-Gp-2′-methyl-C and 2′-methoxy-Gp*-2′-methyl-C

2′-beta-methylcytidine 8 was synthesized based on literature protocols well known in the art, while CE-phosphoramidite 7 is commercially available from various sources. Compound 8 was reacted with DMT-Cl, and benzoyl chloride based on well known procedures. The resultant fully protected cytidine derivative was then treated with p-methylbenzene sulfonic acid to remove the DMT protecting group providing the desired intermediate 9.

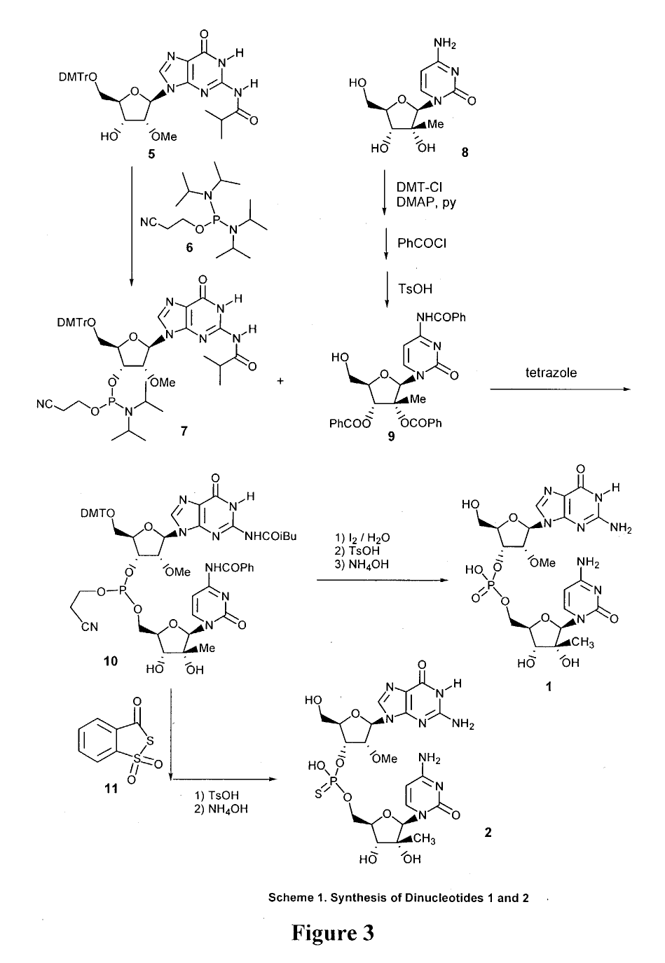

As shown in the scheme of FIG. 3, a mixture of 2′-beta-methyl-2′,3′,4-tribenzoyl-cytidine 9 (3.0 mmol) and tetrazole (0.62 g, 8.7 mmol) was coevaporated 2 times with anhydrous toluene in a dry 100 mL round bottom flask. Anhydrous dichloromethane (20 mL) was added, and the resultant solution was cooled to 0° C. A solution of 3′-EC-phosphoramidite 7 (2.4 mmol) in 8 mL of dichloromethane was added drop wise. The reaction mixture was allowed to warm to room temperature and continue to stir for 3 h. The reaction mixture was diluted with 60 mL of dichloromethane and washed with brine. The aqueous phase was back-extracted with dichloromethane. The combined organic phase was dried over anhydrous sodium sulfate. The solvent was evaporated at room temperature under reduced pressure to give a white foam intermediate 10, which was used for next step without purification.

The resultant white foam was dissolved in 12 mL of THF, and an iodine (0.6 g) solution in THF-water-pyridine (10:5:1) (6.4 mL) was added. The reaction mixture was stirred at room temperature for 15 minutes and concentrated under reduced pressure. The residue was coevaporated with toluene and brought up in 40 mL of chloroform. The solution was washed with 50 mL of 0.2% aqueous sodium bisulfite solution until black color disappeared. The aqueous phase was back-extracted with chloroform. The combined organic phase was added to a solution of p-toluene sulfonic acid (0.6 g) in 30 mL of methanol-dichloromethane (1:1). The mixture was allowed to stir for 20 minutes. 100 mL of ice-cooled saturated sodium bicarbonate was added. The organic phase was separated and washed with water and brine. The organic phase was dried over anhydrous sodium sulfate and concentrated to give 4.6 g of the intermediate as a pale yellow foam. The crude product was purified by flash chromatography on a silica gel column using hexans-acetone (3:2 and 1:2) as eluents to give 2.7 g of white foam. A mixture of this intermediate and 170 mL of concentrated ammonium hydroxide was sealed and stirred at room temperature for 48 h. The reaction mixture was concentrated under reduced pressure. The residue was purified by DEAE-cellulose equilibrated with 0.1 M ammonium bicarbonate solution or purified by HPLC on the reverse phase column to provide the desired dinucleotide 1 as a pale yellow foam in 60% overall yield.

Dinucleotide 2 was synthesized by similar procedure as described above for the synthesis of dinucleotide 1 except compound 11 was used in the place of iodine to oxidize the intermediate phosphorous compound to phosphorothioate linkage.

### Synthesis of 2′-methoxy-Ap-2′-methyl-C and 2′-methoxy-Ap*-2′-methyl-C

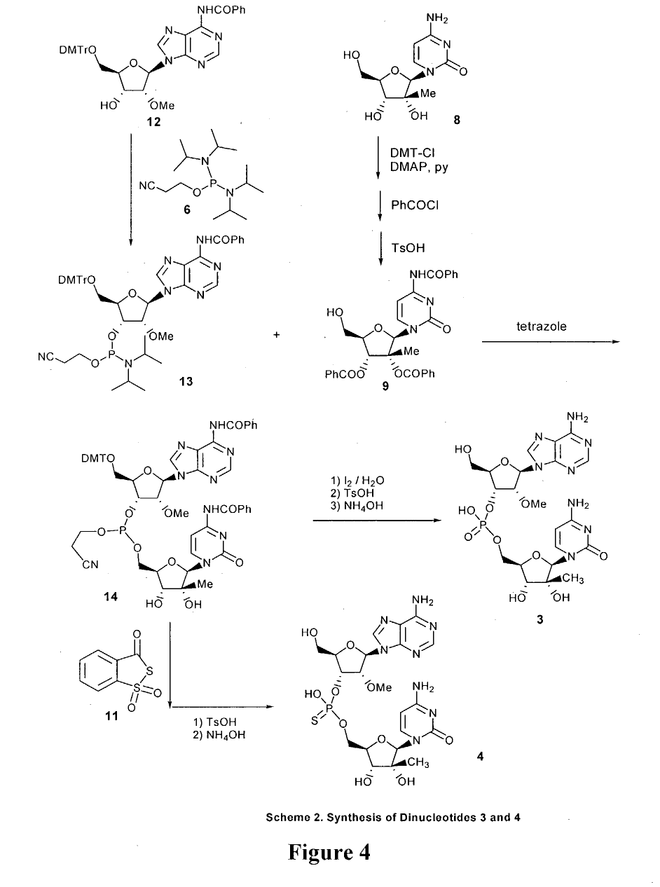

As illustrated in the scheme of FIG. 4, dinucleotides 3 and 4 were synthesized by similar procedures as described above for the synthesis of dinucleotide 1. However, synthesis started from the corresponding adenosine EC-phosphoramidite 13 in place of 7.

### Biological Assays

The inventors discovered that several contemplated compounds exhibited significant antiviral effect, and an especially significant antiviral effect against HCV in vitro and as NS5B inhibitor (Unless indicated otherwise, data are not shown). The assays used to measure the inhibition of HCV NS5B, in vitro cell-based HCV replication, and cytotoxicity are described below.

### Assay of De Novo RNA Synthesis Activity for HCVNS5B Polymerase

All the oligoribonucleosides were purchased from Oligo etc., and were gel-purified. All the chemical reagents were of highest purity possible. H2O used in the assay was RNase and DNase free. [α-33P]-CTP (Ci/mmol) was purchased from ICN Biochemicals or Perkin-Elmer.

A typical assay reaction was carried out at 23° C. for one hour in a buffer containing 20 mM Tris, pH 8.0, 20 mM MgCl2, 10 mM KCl, 5% Glycerol, 5 mM DTT, 0.5 mg/ml BSA, and contemplated compounds at varying concentrations. The template concentration was set at 10 mM and the enzyme concentration at 5 mM. The reaction was quenched by the addition of a loading buffer (80% formamide, 100 mM EDTA, 50 mM Tris borate, 0.15% bromophenol blue and 0.15% of xylene cyanol) and heated to 70° C. for 1 min prior to loading on a 1×TBE polyacrylamide gel. Electrophoresis was performed in 1×TBE at 3000 Volt. Gels were visualized and analyzed by using a PhosphorImager. Unless indicated otherwise, data are not shown for contemplated compounds.

### HCV Replicon Assay

The replicon cells (Huh-7) contain replicating HCV replicon RNA, which was modified in the structural region (replacing the structural region with a neomycin resistance marker). Survival of the replicon cells under G418 selection relies on the replication of HCV RNA and subsequently expression of neomycin phosphoryltransferase. The ability of contemplated compounds to suppress HCV RNA replication was determined using the Quantigene Assay Kit from Bayer. The assay measures the reduction of HCV RNA molecules in the treated cells. Replicon cells were incubated at 37° C. for 3 days in the presence of contemplated compounds before cell harvest for detection. The HCV subgenomic replicon cell line was provided by Dr. Bartenschlager. The assay protocol was modified based on literature procedure (V. Lohmann, F. Korner, J. O. Koch, U. Herian, L. Theilmann, R. Bartenschlager, Science, 1999, 285, 110-113). Unless indicated otherwise, data are not shown for contemplated compounds.

### Cytotoxicity Assay

The cytotoxicity of nucleoside libraries and compounds was measured by the MTS cell based assay from Promega (CellTiter 96 Aqueous One Solution Cell Proliferation Assay). Unless indicated otherwise, data are not shown for contemplated compounds.

Consequently, it is contemplated that the compounds according to the inventive subject matter may be employed in pharmaceutical compositions to treat various viral diseases. In an especially preferred aspect of the inventive subject matter, the inventors contemplate a method of inhibiting replication of a virus in which one or more of the compounds according to the inventive subject matter are provided. In a further step, the virus is presented with the compound(s) at a concentration effective to inhibit replication of the virus. The term “presenting the virus with a compound” as used herein broadly refers to all manners in which the virus or viral component is incubated with the compound.

For example, where the virus or viral component (particularly including a viral RNA dependent RNA polymerase) is in an in vitro system, presentation may comprise admixing the medium in which the virus or viral component is disposed with the compound. In another example, where the virus or viral component is in a cell (either in a cell culture, or in vivo in a hepatocyte in an infected liver of a mammal) it is contemplated that the step of presenting may include administration of a pharmaceutical composition comprising contemplated compounds to the organism in which the virus or viral component is disposed. Suitable pharmaceutical compositions may include oral, parenteral, transdermal, and various other known pharmaceutical compositions. Hence, in an especially preferred aspect, the virus is an HCV virus and is disposed within a cell (which is preferably a hepatocyte in a liver infected with the virus).

Thus, specific embodiments and applications of dinucleotides as specific viral RNA-polymerase inhibitors have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A dinucleotide comprising:
a first nucleoside covalently coupled to a second nucleoside, wherein the dinucleotide inhibits an RNA-dependent RNA polymerase of a virus while the first nucleoside is covalently coupled to the second; and
wherein the at least one of the first nucleoside and the second nucleoside exhibit an antiviral effect against the virus when the dinucleotide is cleaved into a first portion comprising the first nucleoside, and a second portion comprising the second nucleoside.

2. The dinucleotide of claim 1 wherein the first nucleoside is covalently coupled to the second nucleoside via a phosphate group or a modified phosphate group.

3. The dinucleotide of claim 2 further comprising a phosphate group or modified phosphate group covalently coupled to the first nucleoside.

4. The dinucleotide of claim 1 wherein at least one of the first nucleoside and the second nucleoside comprises a 2′-methylribofuranose sugar portion.

5. The dinucleotide of claim 1 wherein the virus is an HCV virus.

6. The dinucleotide of claim 1 having a structure according to Formula 1 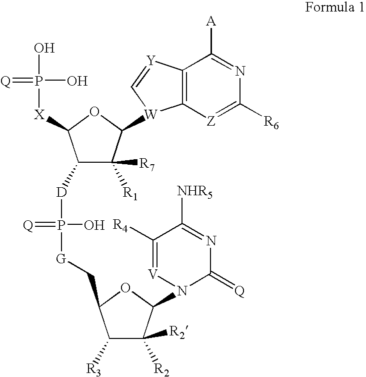
wherein A is H, OR, SR, NH₂, or NHR;
V, W, Y, and Z are independently CH or N;
Q is O or S;
X is O, S, NR, CH₂ or null;
D and G are independently null, CH₂, CF₂, O, S, or NH;
R₁, R₂, R₂′, R₃ are independently H, OR, Halogen, CF₃, CCl₃, CHCl₂, CH₂OH, NO₂, CN, N₃, ═O, SR, NH₂, NHR, NHCOR, NHSO₂R, NHCONHR, or NHCSNHR;
R₄ is R;
R₅ is H, NH₂, NHR, NR₂, NHCOR, NHSO₂R, COR, SO₂R;
R₆ is H, NH₂, NHCOR, NHSR₂R, NHNH₂, NHNHR, NHR, or NR₂;
R₇ is H, OR, SR, Halogen, R, CF₃, CN, CHCl₂, CH₂OH, N₃, NH₂, or CH₂Cl; and
wherein R is H or an optionally substituted alkyl, alkenyl, alkynyl, acyl, or aryl.

7. The dinucleotide of claim 6 wherein R₁, R₂, and R₃ are OH, wherein Q is O, and wherein D and G are O.

8. The dinucleotide of claim 7 wherein at least one of R₂′ and R₇ is CH₃.

9. The dinucleotide of claim 8 wherein W, Y, and Z are N, V is CH, A is NH₂, and wherein R₄, R₅ and R₆ are H.

10. The dinucleotide of claim 6 wherein the 5′-phosphate group is replaced by a OH group.

11. The dinucleotide of claim 6 wherein the 5′-phosphate group is modified such that the modification is preferentially removed from the dinucleotide in a hepatocyte.

12. The dinucleotide of claim 11 wherein the modification comprises a diester.
